(19) United States
(12) Patent Application Publication
(10) Pub. No.: US 2025/0037827 A1
(43) Pub. Date: Jan. 30, 2025

(54) METHODS AND SYSTEMS FOR AUTOMATED WEIGHT-BASED MEDICATION DOSE PREPARATION

(71) Applicant: Nura Medical Inc., Montreal (CA)

(72) Inventors: Georgia Kathleen POWELL, Montreal (CA); Sofia Randa ADDAB, Montreal (CA); Jean-Gabriel LACOMBE, Montreal (CA)

(73) Assignee: Nura Medical Inc., Montreal, QC (CA)

(21) Appl. No.: 18/716,613

(22) PCT Filed: Dec. 7, 2022
(86) PCT No.: PCT/CA2022/051782

§ 371 Date: Jun. 5, 2024

Related U.S. Application Data
(60) Provisional application No. 63/288,060, filed on Dec. 10, 2021.

(51) Int. Cl. G16H 20/10; A61B 5/00; A61B 5/107

(52) U.S. Cl. CPC G16H 20/10; A61B 5/0004; A61B 5/002; A61B 5/1072; A61B 5/7278

(57) ABSTRACT

Various embodiments herein relate to a method and system for automated weight-based medication dose preparation. In at least one embodiment, the system comprises: a weight measuring device comprising: a measuring strip configured to be applied around a patient's limb, the measuring strip movable between an unmounted position and a mounted position; a primary measurement sensor for monitoring movement of the measuring strip into the mounted position; and a device processor coupled to the primary measurement sensor, the device processor operable to: determine, based on data received from the primary measurement sensor, a limb circumference; estimate a patient weight based on the limb circumference; transmit the patient weight to a computing terminal; the computing terminal comprising a computing processor operable to: determine, based on the patient weight and patient-specific data, one or more dosage parameters for a given medication type; and output the one or more dosage parameters.

### CROSS-REFERENCE TO PREVIOUS APPLICATION

This application claims priority from U.S. provisional patent application No. 63/288,060 filed on Dec. 10, 2021, which is incorporated herein by reference in its entirety.

### FIELD

Various embodiments are described herein that generally relate to medication preparation, and in particular, to methods and systems for automated weight-based medication dose preparation.

### BACKGROUND

The following is not an admission that anything discussed below is part of the prior art or part of the common general knowledge of a person skilled in the art.

In clinical settings (i.e., hospital and emergency rooms), health practitioners are often required to administer weight-based medication dosages (i.e., boluses) to patients. To this end, prior to administering the medication dosage, health practitioners will typically complete a weight assessment of the patient, which is then used to perform a weight-based dosage calculation.

It has, however, been appreciated that the timely and accurate calculation, preparation, and administration of weight-based medications presents a major challenge for healthcare professionals, especially in the provision of care for pediatric patients.

In particular, it has been appreciated that errors generally occur at three main points in the process of preparing weight-based medication: (a) estimation of the patient's weight, (b) calculation of the correct volumes of medication and/or diluent, and (c) preparation of the bolus dose, i.e., the syringes and/or or intravenous (IV) bags. Together, these sources of error present a barrier to the delivery of appropriate care and can lead to severe adverse effects for patients.

### SUMMARY OF VARIOUS EMBODIMENTS

Various embodiments of methods and systems for automated weight-based medication dose preparation are provided according to the teachings herein.

According to one broad aspect, there is disclosed a system for weight-based medical dose preparation, the system comprising: a weight measuring device comprising: a measuring strip configured to be applied around a patient's limb, the measuring strip movable between an unmounted position and a mounted position, in which in the mounted position, the strip engages around the patient's limb; at least one motion sensor for monitoring movement of the measuring strip into the mounted position; and a device processor coupled to the at least one motion sensor, the device processor operable to: determine, based on data received from the at least one motion sensor, a limb circumference; estimate a patient weight based on the limb circumference; and transmit the patient weight to a computing terminal, the computing terminal comprising a computing processor operable to: receive the patient weight from the weight measuring device; determine, based on the given medication type and one or more of the patient weight and patient-specific data, one or more dosage parameters; and output the one or more dosage parameters.

In some embodiments, the weight measuring device is located remotely from the computing terminal, and the system further comprises a communication network, and wherein, the weight measuring device further comprises a device communication interface coupled to the device processor, and the device processor being further configured to transmit the patient weight over the communication network, via the device communication interface; and the computing terminal further comprises a computing communication interface coupled to the computing processor, and the computing processor is operable to receive the patient weight via the computing communication interface.

In some embodiments, the communication network comprises a personal area network (PAN).

In some embodiments, the weight measuring device and the computing terminal are directly coupled to form a single integrated device.

In some embodiments, the at least one motion sensor comprises an encoder unit.

In some embodiments, the measuring strip is applied around one of the patient's middle-upper arm and the patient's thigh.

In some embodiments, the dosage parameters comprise one or more of a suggested dose, a total dose, and a drug volume.

In some embodiments, the patient-specific data comprises one or more patient age and patient gender.

In some embodiments, estimating the patient weight based on the limb circumference comprises: identifying a circumference range for the limb circumference; and applying a predictive model associated with the identified circumference range.

In some embodiments, estimating the patient weight based on the limb circumference comprises: determining a patient age range; and applying the predictive model associated with the identified circumference range and the patient age range.

According to another broad aspect, there is disclosed a method for weight-based medical dose preparation for a given medication type, comprising: determining, based on data received from at least one motion sensor of the weight measuring device, a limb circumference, wherein the at least one motion sensor monitors movement of a measuring strip applied around a patient's limb in a mounted position; estimating a patient weight based on the limb circumference; determining, based on the given medication type and one or more of the patient weight and patient-specific data, one or more dosage parameters; and outputting the one or more dosage parameters.

In some embodiments, the method further comprises: transmitting the patient weight over a communication network from the weight measuring device to a remote computer terminal; and receiving the patient weight at the computing terminal.

In some embodiments, the communication network comprises a personal area network (PAN).

In some embodiments, the weight measuring device and the computing terminal are directly coupled to form a single integrated device.

In some embodiments, the at least one motion sensor comprises an encoder.

In some embodiments, the measuring strip is applied around one of the patient's middle-upper arm and the patient's thigh.

In some embodiments, the dosage parameters comprise one or more of a suggested dose, a total dose, and a drug volume.

In some embodiments, the patient-specific data comprises one or more of patient age and patient gender.

In some embodiments, estimating the patient weight based on the limb circumference comprises: identifying a circumference range for the limb circumference; and applying a predictive model associated with the identified circumference range.

In some embodiments, estimating the patient weight based on the limb circumference comprises: determining a patient age range; and applying the predictive model associated with the identified circumference range and the patient age range.

According to another broad aspect, there is disclosed a weight measuring device comprising: a measuring strip configured to be applied around a patient's limb, the measuring strip movable between an unmounted position and a mounted position, in which in the mounted position, the strip engages around a patient's limb; at least one motion sensor for monitoring movement of the measuring strip into the mounted position; and a device processor coupled to the at least one motion sensor, the device processor operable to: determine, based on data received from the at least one motion sensor, a limb circumference; estimate a patient weight based on the limb circumference; and output the estimated patient weight.

In some embodiments, the at least one motion sensor comprises an encoder unit.

In some embodiments, the measuring strip is applied around one of the patient's middle-upper arm and the patient's thigh.

In some embodiments, estimating the patient weight based on the limb circumference comprises: identifying a circumference range for the limb circumference; and applying a predictive model associated with the identified circumference range.

In some embodiments, the patient weight based on the limb circumference comprises: determining a patient age range; and applying the predictive model associated with the identified circumference range and the patient age range.

According to another broad aspect, there is provided a system for weight-based medical dose preparation. The system comprises a weight measuring device. The weight-based measuring device comprises a measuring strip configured to be applied around a patient's limb, the measuring strip movable between an unmounted position and a mounted position, in which in the mounted position, the strip engages around the patient's limb. The weight-based measuring device comprises a primary measurement sensor for measuring limb circumference using the measuring strip. The weight-based measuring device comprises a device processor coupled to the primary measurement sensor. The device processor operable to determine, based on data received from the primary measurement sensor, a limb circumference. The device processor is also operable to estimate a patient weight based on the limb circumference and transmit the patient weight to a computing terminal. The computing terminal comprises a computing processor operable to receive the patient weight from the weight measuring device. The computing processor is also operable to determine, based on the given medication type and one or more of the patient weight and patient-specific data, one or more dosage parameters and output the one or more dosage parameters.

In some embodiments, the weight measuring device is located remotely from the computing terminal, and the system further comprises a communication network. The weight measuring device further comprises a device communication interface coupled to the device processor, and the device processor is further configured to transmit the patient weight over the communication network, via the device communication interface. The computing terminal further comprises a computing communication interface coupled to the computing processor, and the computing processor is operable to receive the patient weight via the computing communication interface.

In some embodiments, the communication network comprises a personal area network (PAN).

In some embodiments, the weight measuring device and the computing terminal are directly coupled to form a single integrated device.

In some embodiments, the primary measurement sensor comprises an encoder unit.

In some embodiments, the measuring strip is applied around one of the patient's middle-upper arm and the patient's thigh.

In some embodiments, the dosage parameters comprise one or more of a suggested dose, a total dose, and a drug volume.

In some embodiments, the patient-specific data comprises one or more patient age and patient gender.

In some embodiments, estimating the patient weight based on the limb circumference comprises identifying a circumference range for the limb circumference and applying a predictive model associated with the identified circumference range.

In some embodiments, estimating the patient weight based on the limb circumference comprises determining a patient age range and applying the predictive model associated with the identified circumference range and the patient age range.

According to another broad aspect, there is provided a method for weight-based medical dose preparation for a given medication type. The method comprises determining, based on data received from a primary measurement sensor of the weight measuring device, a limb circumference. The primary measurement sensor measures limb circumference using the measuring strip. The method further comprises estimating a patient weight based on the limb circumference, determining, based on the given medication type and one or more of the patient weight and patient-specific data, one or more dosage parameters, and outputting the one or more dosage parameters.

In some embodiments, the method further comprises transmitting the patient weight over a communication network from the weight measuring device to a remote computer terminal and receiving the patient weight at the computing terminal.

In some embodiments, the communication network comprises a personal area network (PAN).

In some embodiments, the weight measuring device and the computing terminal are directly coupled to form a single integrated device.

In some embodiments, the primary measurement sensor comprises an encoder.

In some embodiments, the measuring strip is applied around one of the patient's middle-upper arm and the patient's thigh.

In some embodiments, the dosage parameters comprise one or more of a suggested dose, a total dose, and a drug volume.

In some embodiments, the patient-specific data comprises one or more of patient age and patient gender.

In some embodiments, estimating the patient weight based on the limb circumference comprises identifying a circumference range for the limb circumference, and applying a predictive model associated with the identified circumference range.

In some embodiments, estimating the patient weight based on the limb circumference comprises determining a patient age range and applying the predictive model associated with the identified circumference range and the patient age range.

According to another broad aspect, there is provided a weight measuring device. The weight measuring device comprises a measuring strip configured to be applied around a patient's limb, the measuring strip is movable between an unmounted position and a mounted position, in which in the mounted position, the strip engages around a patient's limb. The weight measuring device comprises a primary measurement sensor for monitoring movement of the measuring strip into the mounted position. The weight measuring device further comprises a device processor coupled to the primary measurement sensor. The device processor is operable to, determine, based on data received from the primary measurement sensor, a limb circumference, estimate a patient weight based on the limb circumference, and output the estimated patient weight.

In some embodiments, the primary measurement sensor comprises an encoder unit.

In some embodiments, the measuring strip is applied around one of the patient's middle-upper arm and the patient's thigh.

In some embodiments, estimating the patient weight based on the limb circumference comprises identifying a circumference range for the limb circumference, and applying a predictive model associated with the identified circumference range.

In some embodiments, estimating the patient weight based on the limb circumference comprises determining a patient age range and applying the predictive model associated with the identified circumference range and the patient age range.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1A shows an example embodiment of an environment in which the methods and systems disclosed herein may operate;

FIG. 1B shows another example embodiment of an environment in which the methods and systems disclosed herein may operate;

FIG. 2 is a system for automated weight-based medication dose preparation, according to some example embodiments;

FIG. 3A is an example graphical user interface (GUI) displaying an introductory, or welcome screen associated with an automated medication dose preparation software;

FIG. 3B is an example GUI associated with the automated medication dose preparation software, and displaying fields to be populated with patient-specific data;

FIG. 3C is an example GUI associated with the automated medication dose preparation software, and displaying options for medication selection;

FIG. 3D is an example GUI associated with the automated medication dose preparation software, and displaying suggested medication dosage;

FIG. 3E is an example GUI associated with the automated medication dose preparation software, and displaying suggested medication dosage parameters;

FIG. 3F is an example GUI associated with the automated medication dose preparation software, and displaying further suggested medication dosage parameters;

FIG. 4A is a front perspective view of an example embodiment of a weight estimation device;

FIG. 4B is a front elevation view of the weight estimation device;

FIG. 4C is a rear perspective view of the weight estimation device;

FIG. 4D is a rear cross-sectional perspective view of the weight estimation device, taken along section line 4-4′ in FIG. 4A;

FIG. 4E is a rear cross-sectional elevation view of the weight estimation device, taken along section line 4-4′ in FIG. 4A;

FIG. 4F is another rear cross-sectional perspective view of the weight estimation device, taken along section line 4-4′ in FIG. 4A;

FIG. 5 is a simplified block diagram of electrical hardware components in an example weight estimation device and an example computer terminal;

FIG. 6 is a process flow for an example embodiment of a method for dose preparation;

FIG. 7 is a process flow for an example embodiment of a method for automated weight estimation;

FIG. 8A is an example plot showing the relationship between arm circumference and body weight for subjects within an age range of 0 to 4 years old; and

FIG. 8B is an example plot showing the relationship between arm circumference and body weight for subjects within an age range of 4 to 19 years old.

Further aspects and features of the example embodiments described herein will appear from the following description taken together with the accompanying drawings.

### DESCRIPTION OF VARIOUS EMBODIMENTS

Various systems or methods will be described below to provide an example of an embodiment of the claimed subject matter. No embodiment described below limits any claimed subject matter and any claimed subject matter may cover methods or systems that differ from those described below. The claimed subject matter is not limited to systems or methods having all of the features of any one system or method described below or to features common to multiple or all of the apparatuses or methods described below. It is possible that a system or method described below is not an embodiment that is recited in any claimed subject matter. Any subject matter disclosed in a system or method described below that is not claimed in this document may be the subject matter of another protective instrument, for example, a continuing patent application, and the applicants, inventors or owners do not intend to abandon, disclaim or dedicate to the public any such subject matter by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

It should also be noted that the terms “coupled” or “coupling” as used herein can have several different meanings depending in the context in which these terms are used. For example, the terms coupled or coupling may be used to indicate that an element or device can electrically, optically, or wirelessly send data to another element or device as well as receive data from another element or device. As used herein, two or more components are said to be “coupled”, or “connected” where the parts are joined or operate together either directly or indirectly (i.e., through one or more intermediate components), so long as a link occurs. As used herein and in the claims, two or more parts are said to be “directly coupled”, or “directly connected”, where the parts are joined or operate together without intervening intermediate components.

It should be noted that terms of degree such as “substantially”, “about” and “approximately” as used herein mean a reasonable amount of deviation of the modified term such that the end result is not significantly changed. These terms of degree may also be construed as including a deviation of the modified term if this deviation would not negate the meaning of the term it modifies.

Furthermore, any recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

The example embodiments of the systems and methods described herein may be implemented as a combination of hardware or software. In some cases, the example embodiments described herein may be implemented, at least in part, by using one or more computer programs, executing on one or more programmable devices comprising at least one processing element, and a data storage element (including volatile memory, non-volatile memory, storage elements, or any combination thereof). These devices may also have at least one input device (e.g. a pushbutton keyboard, mouse, a touchscreen, and the like), and at least one output device (e.g. a display screen, a printer, a wireless radio, and the like) depending on the nature of the device.

It should also be noted that there may be some elements that are used to implement at least part of one of the embodiments described herein that may be implemented via software that is written in a high-level computer programming language such as object-oriented programming or script-based programming. Accordingly, the program code may be written in Java, Swift/Objective-C, C, C++, Javascript, Python, SQL or any other suitable programming language and may comprise modules or classes, as is known to those skilled in object-oriented programming. Alternatively, or in addition thereto, some of these elements implemented via software may be written in assembly language, machine language or firmware as needed. In either case, the language may be a compiled or interpreted language.

At least some of these software programs may be stored on a storage media (e.g. a computer readable medium such as, but not limited to, ROM, magnetic disk, optical disc) or a device that is readable by a general or special purpose programmable device. The software program code, when read by the programmable device, configures the programmable device to operate in a new, specific and predefined manner in order to perform at least one of the methods described herein.

Furthermore, at least some of the programs associated with the systems and methods of the embodiments described herein may be capable of being distributed in a computer program product comprising a computer readable medium that bears computer usable instructions for one or more processors. The medium may be provided in various forms, including non-transitory forms such as, but not limited to, one or more diskettes, compact disks, tapes, chips, and magnetic and electronic storage. The computer program product may also be distributed in an over-the-air or wireless manner, using a wireless data connection.

The term “software application” or “application” refers to computer-executable instructions, particularly computer-executable instructions stored in a non-transitory medium, such as a non-volatile memory, and executed by a computer processor. The computer processor, when executing the instructions, may receive inputs and transmit outputs to any of a variety of input or output devices to which it is coupled. Software applications may include mobile applications or “apps” for use on mobile devices such as smartphones and tablets or other “smart” devices.

A software application can be, for example, a monolithic software application, built in-house by the organization and possibly running on custom hardware; a set of interconnected modular subsystems running on similar or diverse hardware; a software-as-a-service application operated remotely by a third party; third party software running on outsourced infrastructure, etc. In some cases, a software application also may be less formal, or constructed in ad hoc fashion, such as a programmable spreadsheet document that has been modified to perform computations for the organization's needs.

Software applications may be deployed to and installed on a computing device on which it is to operate. Depending on the nature of the operating system and/or platform of the computing device, an application may be deployed directly to the computing device, and/or the application may be downloaded from an application marketplace. For example, user of the user device may download the application through an app store such as the Apple App Store™ or Google™ Play™.

Current methods for weight-based medical dose preparation often suffer from a number of significant drawbacks. For instance, in many cases, weight-based dose preparation may be manually performed by health care practitioners—i.e., nurses in emergency departments, or by a centralized or de-centralized pharmacy team in other hospital units. In particular, health practitioners may initially weight the subject patient, and may then perform manual calculations to determine the appropriate medication dosage. Manual determinations of this nature are, however, subject to significant human-error, and may yield inaccurate results that prove detrimental to the patient's health. In developing countries, the problems are even more acute, as medical facilities often lack scales as well as other basic tools required to accurately assess patient weight.

In recent years, there have been some attempts at automating the process of weight-based dose calculations using tools such as smartphone applications and computer software. However, many software solutions require manual input of patient data (i.e., thereby increasing likelihood of input errors), often lack in reliability and can be inaccessible to health staff. Applications and software solutions also do not offer case-dependent preparation support, and do not assist in the prioritization of medications or identification of potential adverse drug interactions. Additionally, current automated IV medication preparation software systems focus primarily on oncology or on large-scale pharmaceutical use.

It has been further appreciated that, in the special context of pediatrics—current available tools utilize recumbent length and/or age of the child as a predictor of weight. These approaches have been shown to perform well only in specific subsets of the population and do not address body composition and ethnic differences. Moreover, many of the methods utilizing length are not appropriate for tall children and young teenagers. Still further, many current solutions do not allow for real-time or near real-time monitoring of pediatric patients' weight. This has particular significance in pediatric ICUs, where all medication is weight-based—accordingly, having an updated and accurate weight is critical for providing pediatric patients with the correct medication dose

Still further yet, the inventors have appreciated that there are no existing effective tools which can, in addition to automatically determining appropriate weight-based bolus dosage, can also automatically prepare the bolus dose, i.e., automatically prepare a syringe or IV bag. If present, such tools would have significance in eliminating, or reducing, errors in the bolus preparation process.

In view of the foregoing, to mitigate at least some of the aforementioned drawbacks of conventional methods, embodiments herein provide for a method and system for automated weight-based medication preparation.

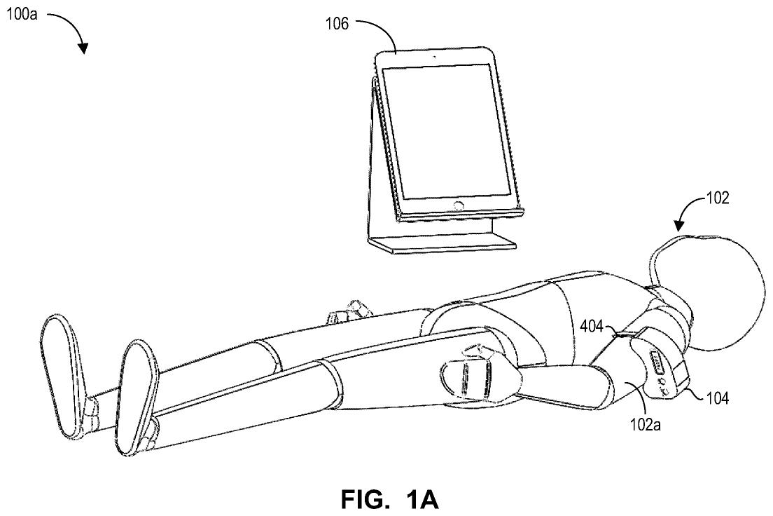

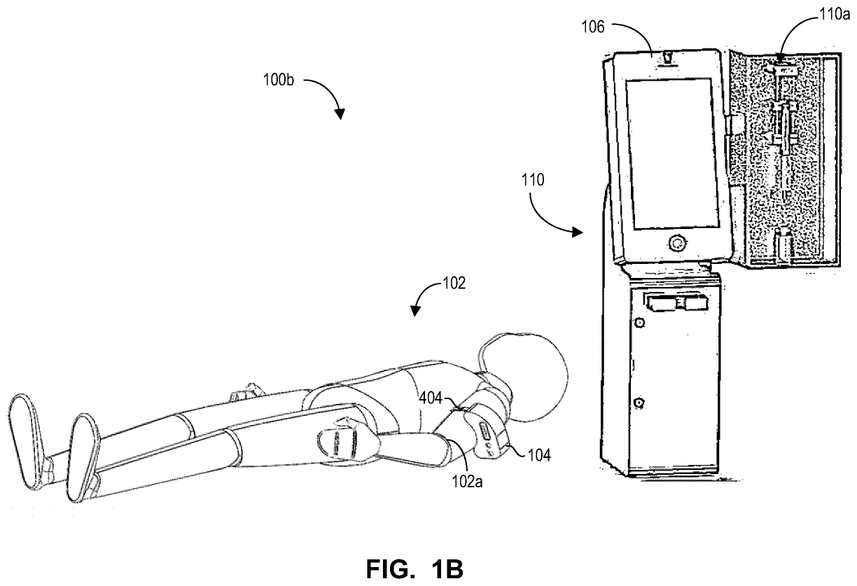

Reference is now made to FIG. 1A, which shows an example environment 100a in which the methods and systems disclosed herein may operate, in accordance with some embodiments.

As shown, a patient 102 may be located in a medical or clinical setting, and may require delivery of a weight-based dose medication, i.e., orally, via a syringe, IV line or interosseous infusion, etc. Patient 102 may be, for example, a pediatric patient.

To mitigate for at least some of the aforementioned challenges in accurate estimation of the patient's weight, embodiments herein provide for a limb-based weight estimation device 104. The limb-based weight estimation device 104 can be used for real-time or near real-time weight estimation.

As shown, the weight estimation device 104 may be applied around the subject's limb to automatically measure limb circumference. As provided herein, the measured limb circumference is used as a surrogate to estimate the patient's weight. In the illustrated embodiment, the weight estimation device 104 is applied around the subject's arm 102a to measure the middle-upper arm circumference. In other cases, however, the device 104 may also be applied around the subject's thigh to measure thigh circumference, around the subject's calf to measure calf circumference or otherwise any other suitable part of the arm or leg. In any case, the weight estimation device 104 can be applied to the patient, irrespective of the position of the patient (i.e., standing, sitting or lying down). This may be particularly advantageous for patients, i.e., pediatric patients, who are immobile.

In at least some embodiments, the device 104 may, itself, automatically determine the subject's weight based on the measured limb circumference using a circumference-based weight prediction algorithm. The estimated weight data is then communicated to an external computing device 106 to determine weight-based medication dosage for a given medication type. In other embodiments, device 104 may determine and transmit the patient limb circumference data, i.e., to the external computing device 106. The external computing device 106 may then estimate subject's weight.

To this end, the concept of using limb circumference as an estimate of weight is premised on the notion that surrogate limb circumference measurements generally introduce less error in weight prediction than exiting methods that rely on other factors, such as recumbent length and/or subject age. The use of limb circumference also significantly extends the age range for which weight estimates can be obtained through use of surrogate measurement. Additionally, the use of the automated device 104 increases repeatability and reliability of measured surrogate measurements, and simplifies the measurement process. The automated weight estimation device 104 also significantly reduces rate of human error from user-dependent weight estimates. As the weight estimation data is automatically communicated to computing terminal 106, this can also further reduce the risk of human error by removing the need for manual input of patient data into applications and/or software systems operating on terminal 106.

External computing terminal 106 may host a medication dose preparation software. The medication dose software is operable to perform weight-to-medication computation. The weight-to-medication computation converts, weight estimation data—i.e., received from the device 104, as well as other patient-specific data (i.e., gender, age, etc.), to correct medication therapeutic dosage ranges for a given medication. In various cases, this conversion is performed using pre-defined medication libraries, which provide optimal, or standard, medication dosage (mg/kg) for different patient weights and for different medication types.

In some embodiments, as stated previously, computing terminal 106 may not receive weight estimation data from device 104, but may only receive limb circumference data. In other words, rather than estimating weight—the weight estimation device 104 may only transmit limb circumference data to terminal 106. In these cases, prior to performing weight-to-medication computation, the software may initially estimate patient weight from received limb circumference data.

In at least one embodiment, the medication dose software—operating on computing terminal 106—may present various procedural information for preparing and administering medication. In some cases, where multiple medication types are administrated to the patient, the medication dose software can also enable automatic prioritization of administered medications according to standard of care algorithms. The software may also detect pharmaceutical interactions between multiple medication types, and may warn the health practitioner of adverse effects. This can be performed using pre-defined clinical treatment flows and/or via aggregation of data and statistical predictions according to historical treatment flows. In general, the collective of these features reduces the need for manual calculations and provide further insight to healthcare professionals on the preparation of time-sensitive medications.

While the illustrated embodiment has shown the computing terminal 106 and weight estimation device 104 as being separate components—it will be understood that in other embodiments, these may in-fact be a single integrated component. For example, the weight estimation device 104 may include integrated computing functionality for hosting the medication dose software.

In view of the foregoing discussion, it will now be appreciated that the weight estimation device 104 and the medication dose software—operating on computing terminal 106—taken alone or in combination, have a number of useful applications.

In one example application, the combination of the weight estimation device 104 and medication dose software may be applied in a clinical setting, such as an emergency room. For instance, a pediatric patient having an unknown weight, and in critical condition, may be presented to an emergency department. Upon presentation, healthcare practitioners may immediately apply the weight estimation device 104 on the patient, and may use the medication dose software—on computer terminal 106—to determine appropriate dosage parameters (e.g., suggested dosage, total dosage, drug volume, etc.) for one or more medications prescribed by an attending physician. In addition to determining dosage, the medication dose software may also provide guiding instructions for preparing and administering the medication, as well as providing guidance on the prioritization order of multiple administered medication doses. Accordingly, in this example case, the concurrent use of the weight estimation device 104 and medication dose software provides for accurate determination and administration of medication doses in a time-sensitive manner.

In another example application, the weight estimation device 104 and medication dose software may be used in intensive care units (ICU). In ICUs, obtaining a patient's accurate weight can present a challenge as the majority of patients are immobile and are connected to various medical equipment, such as IV lines and oxygen masks. The weight estimation device 104 accordingly provides a convenient solution for measuring weight of patients in an immobile state. Additionally, the weight estimation device 104 may have practical benefit for real-time or near real-time monitoring of a pediatric patient's weight, or otherwise any patient's weight. In particular, in pediatric ICUs, all medications are weight-based, therefore having an updated and accurate weight is critical for providing pediatric patients with the correct medication dose. Currently, based on patient and hospital, patient weight may only be measured on an intermittent basis, i.e., weekly basis.

In yet another example application, the weight estimation device 104 and medication dose software can be used remotely from one other. For example, the weight estimation device 104 may be available in an ambulance, while the medication dose software may be operating on a computer terminal 106 located at a receiving hospital. Typically, paramedics and various emergency response teams have limited scope in their ability to perform life-saving measures. Accordingly, as the patient is being transported to the hospital, it is vital that nurses and physicians (i.e., at the receiving hospital) have advanced opportunity to prepare for the incoming trauma. In these circumstances, as the patient is on route to the hospital, the paramedics can apply the weight estimation device 104, and may contact healthcare professionals in the emergency room to relay weight estimation data displayed on the device 104. In turn, health care professionals can begin advanced preparation of the weight-based medication, i.e., using the medication dose software operating on computer terminal 106, in anticipation of the patient's arrival. In this manner, the medication is readily prepared well in advance of the patient's arrival at the hospital. In other cases, rather than the paramedic verbally communicating weight estimation data to the healthcare team at the hospital—weight estimation data may also be automatically communicated by the weight estimation device 104 to the computing terminal 106 located at the hospital (i.e., via satellite or internet networks).

In some example applications, the weight estimation device 104, and the medication dose software—operating on the computing terminal 106—may be used independently.

For instance, during triage in an emergency department, the weight estimation device 104 may be applied independently to the patient upon arrival (i.e., a pediatric patient). A triage nurse may begin by assessing the patient's weight using the weight estimation device 104. The nurse may remove the device from the patient's arm, and the weight will remain on the screen of the device. The nurse may then record the weight in the hospital's electronic medical records system or on a paper chart and can continue their assessment of the patient

In another example, the medication dose software may also be used independently from the weight estimation device 104, as for example in non-urgent or non-critical clinical settings. For instance, if the patient's weight is already known, a health care professional may enter the patient's weight directly into the medication dose software, without the use of the weight estimation device 104. The medication dose software may then determine the appropriate medication dose for a given medication, as well as provide guiding instructions for administering the dose.

Reference is now made to FIG. 1B, which shows another example environment 100b in which the methods and systems disclosed herein may operate, according to some other embodiments.

The environment 100b is generally analogous to the environment 100a, with the exception that the computing terminal 106 is now coupled to a bolus dose preparation system 110. Bolus dose preparation system 110 is operable to automatically prepare a bolus dose based on dosage parameters determined, for example, by the medication dose software. In various cases, the medication dose software (or some other software) may be operable to control hardware components of the bolus dose preparation system 110 to automatically fill a syringe 110a or an IV bag. This, in turn, eliminates or reduces errors associated with the actual bolus dose preparation process. In some cases, a separate computing device—which may be in communication with computing terminal 106—may control the dose preparation system 110.

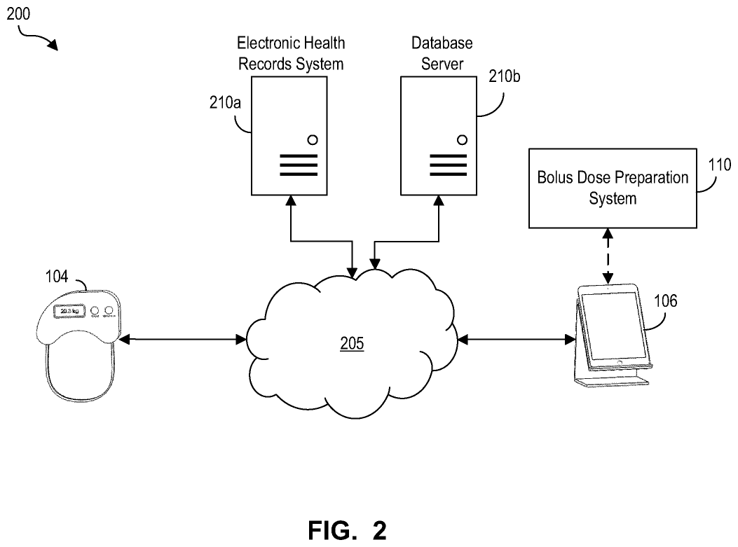

Reference is now made to FIG. 2, there is shown a system 200 for automated weight-based medication dose preparation, in accordance with some embodiments.

As shown, system 200 generally includes the weight estimation device 104 connected, via network 205, to external computing terminal 106. In some embodiments, external computing terminal 106 may itself be connected to the bolus dose preparation system 110, as explained with reference to FIG. 1B. In some cases, system 200 further includes one or more servers 210 connected to the network 205.

Network 205 may be any network capable of carrying data include any wired or wireless network. By way of non-limiting examples, network 205 may comprise an Internet network, Ethernet network, plain old telephone service (POTS) line, public switch telephone network (PSTN), integrated services digital network (ISDN), digital subscriber line (DSL), coaxial cable, fiber optics, satellite, mobile, wireless (e.g., Wi-Fi, WiMAX), SS7 signaling network, fixed line, local area network, wide area network, and others, including any combination of these. In some cases, network 205 may be constructed from one or more computer network technologies, such as IEEE 802.3 (Ethernet), IEEE 802.11 and similar technologies. In other cases, network 205 may comprise a personal area network (PAN), such as a Bluetooth® connection. In some cases, network 205 may comprise a combination of multiple network types. For example, the communication between device 104 and computer 106 may occur over Bluetooth® connection, while the communication between device 104/computer 106 and servers 210 may occur over a non-Bluetooth® connection.

External computer terminal 106 may be any suitable computing device capable of executing software programs and applications. For example, computer terminal 106 may be a tablet computer, a smartphone, desktop or laptop computer, as well as a wide variety of “smart” devices capable of data communication. Computer terminal 106 may be portable, and may at times be connected to network 205 or a portion thereof.

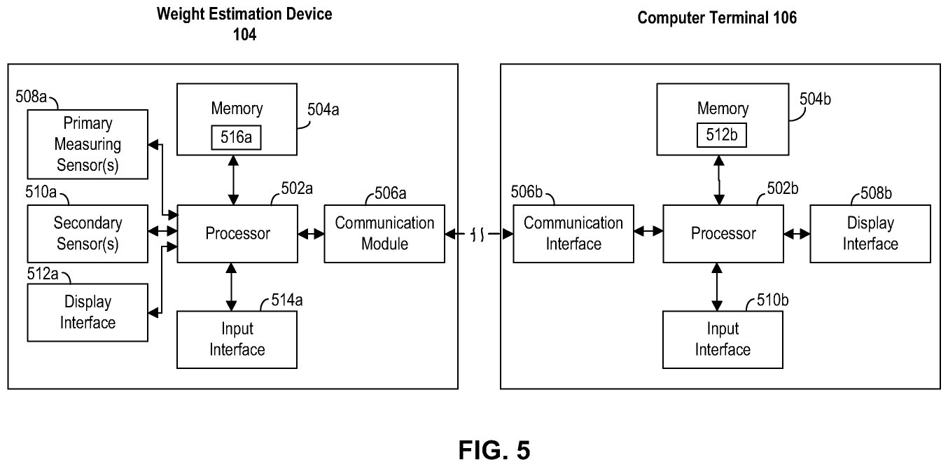

As provided in greater detail herein, with reference to FIG. 5, computer terminal 106 may include a processor 502b coupled, via a data bus, to a volatile and non-volatile memory 504b, at least one communication interface 506b, a display interface 508b and an input interface 510b.

In at least one embodiment, memory 502b—of computing terminal 106—may store the medication dose preparation software 512b. As explained previously, among other functionalities, the medication dose preparation software 512b may be operable to automatically determine bolus doses using estimated patient weight data (as well as other patient-specific data). Medication dose preparation software 512b may also detect various pharmaceutical interactions to generate warnings of adverse effects. In still other cases, medication dose preparation software 512b may be operable to control the medication dose preparation system 110.

Servers 210 are computer servers that are connected to network 205. Similar to computing terminal 106, servers 210 may also have a processor, volatile and non-volatile memory, at least one network interface, and may have various other input/output devices.

As with all devices shown in the system 200, there may be multiple servers 210, although not all are shown. It will be understood that the server 210 need not be a dedicated physical computer. For example, in various embodiments, the various logical components that are shown as being provided on server 210 may be hosted by a third party “cloud” hosting service such as Amazon™ Web Services™ Elastic Compute Cloud (Amazon EC2).

In at least one embodiment, servers 210 can include an electronic health records server 210a and/or a database server 210b. Electronic health records server 210a may include various databases for storing patient-specific data (i.e., patient IDs, gender, etc.) In some cases, electronic health records server 210a can be associated, for example, with a third-party, such as a local health service department.

Database server 210b can be used to store various databases for access by the medication preparation software 512b. For example, the database server may store pre defined medication libraries that can be accessed to compute weight-based medication dosages. Database server 210b can also store pre-defined medication interaction data that can be used to detect pharmaceutical interactions. In other cases, this data may be stored locally on the memory 502b of the computer terminal 106.

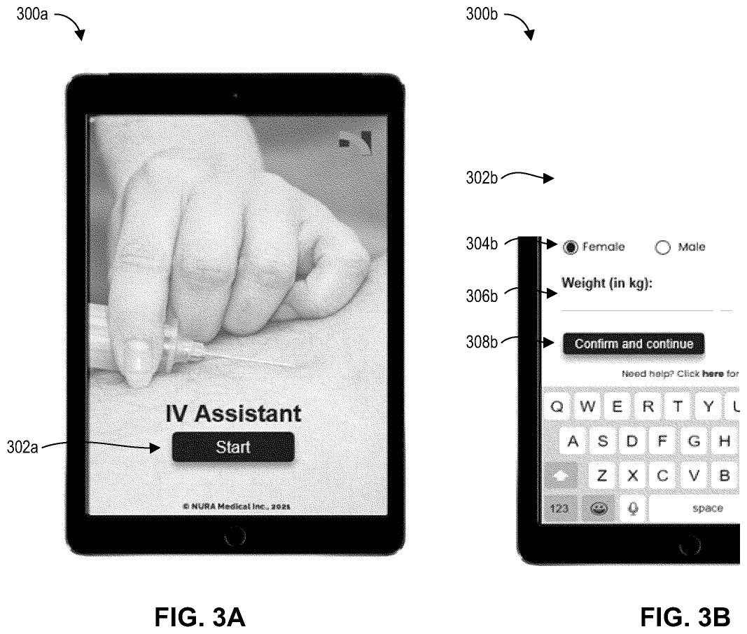

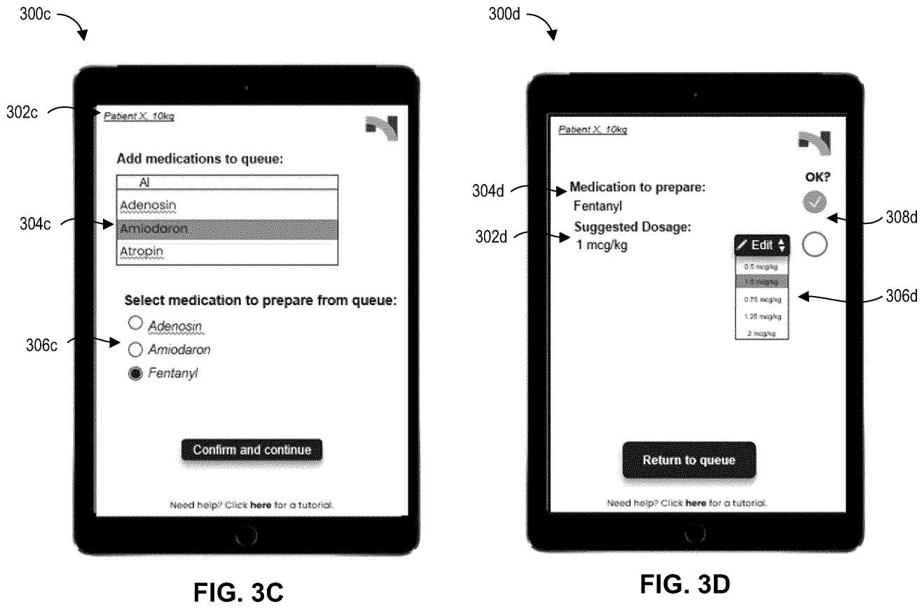

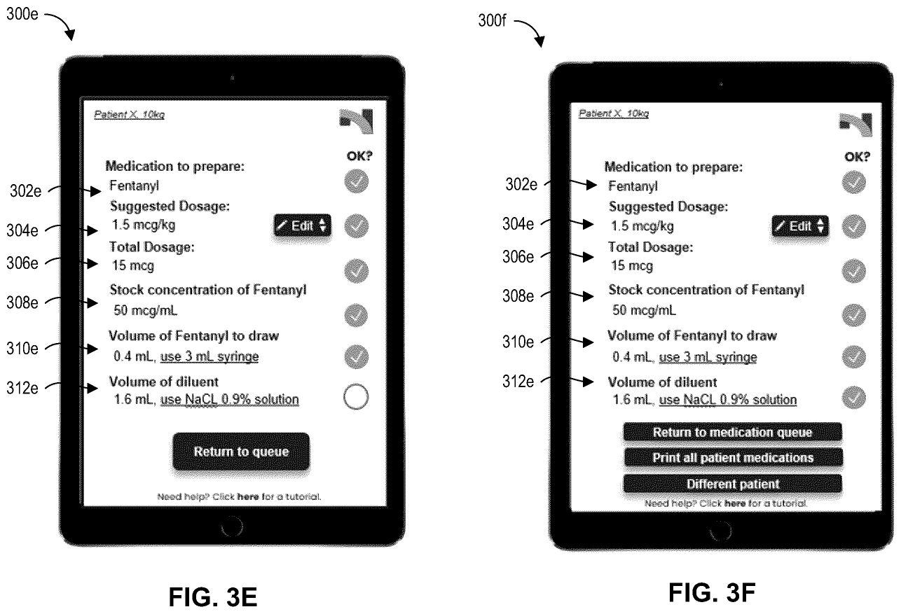

Reference is now made to FIGS. 3A-3F, which show various screenshots of example graphical user interfaces (GUIs) that may be displayed on the computer terminal 106. The GUIs may be displayed, for example, on the computer's display interface 508b, and may be generated by the bolus dose preparation software 512b executing on processor 502a (FIG. 5).

FIG. 3A illustrates an example screenshot of a GUI 300a which displays an introductory, or welcome screen. The introductory screen enables a user (i.e., a health practitioner) to select a “start” option 302a to commence medication dose preparation.

FIG. 3B illustrates an example screenshot of a GUI 300b that may follow the introductory screen 300a. Screen 300b prompts the user to enter patient-specific data. For example, the patient-specific data can include a patient ID 302b, the patient's sex 304b as well as the patient's weight 306b.

In some embodiments, rather than inputting patient-specific data, the software 506 may request—or automatically receive—the data from the electronic health records server 210a. For example, the user may enter the patient ID, and the software 506 may use the patient ID to retrieve associated patient-specific data that is pre-stored on the server 210a.

In other cases, once the user has entered the patient-specific data into the screen 300b, the software 506 may automatically communicate this data to the server 210a for recording. In some cases, this communication may occur in real-time, or near real-time, to ensure patient records on server 210a are up-to-date. In any case, once patient-specific data is entered or retrieved, the user may confirm by selecting option 308b.

FIG. 3C illustrates an example screenshot of a further GUI 300c. The GUI screen 300c may display the estimated patient weight 302c (i.e., 10 Kg). The estimated patient weight can be determined based on data received from the weight estimation device 104. For example, before, after or concurrently while the patient-specific data is being entered (or otherwise being externally received from server 210a)—the device 104 may be applied to the patient 102. The device 104 may then automatically determine estimated patient weight based on measured limb circumference. Device 104 may then transmit (automatically or upon request) the estimated weight to the computer terminal 106.

In other cases, the device 104 may not estimate patient weight, and may only determine limb circumference data. In these cases, only limb circumference data is transmitted from the device 104 to the software 512b. Software 512b may then, itself, apply a circumference-based weight prediction algorithm to estimate patient weight.

As shown, screen 300c may also display an option feature 304c to enable selection between multiple types of medication to queue for administering to a patient. Option 306c enables the user to select one of the queued medications to administer to the patient.

FIG. 3D illustrates an example screenshot of a GUI 300d in which software 512b may automatically determine a suggested bolus dosage 302d for the selected medication 304d (i.e., 1 mcg/kg of Fentanyl). In embodiments provided herein, this may be determined based on analyzing the patient's estimated weight 302c, as well as the patient-specific data.

In some cases, software 512b may have access to pre-defined medication libraries that can be used to determine medication dosage based on patient weight. The medication libraries may be stored directly on the computer terminal 106, or may be accessed (or retrieved) from external server 210b. In some cases, the user may also have the option to edit or revise the dosage at 306d.

FIG. 3E shows an example screen 300e in which the software 512b may present the user with summary details in respect of the medication to prepare (i.e., Fentanyl) 302e, the suggested dosage (1.5 mcg/kg) 304e, total dosage (15 mcg) 306e, stock concentration 308e, volume of medication to draw 310e and volume of diluent 312e. In various cases, total dosage (mg) 306e may be determined as the dosage (mg/kg)×patient weight (kg). Further, the volume to draw from stock medication vial 310e may be determined by total dosage (mg)/medication stock concentration (mg/mL). In FIGS. 3E and 3F, the user may have the option to confirm each of these selection parameters.

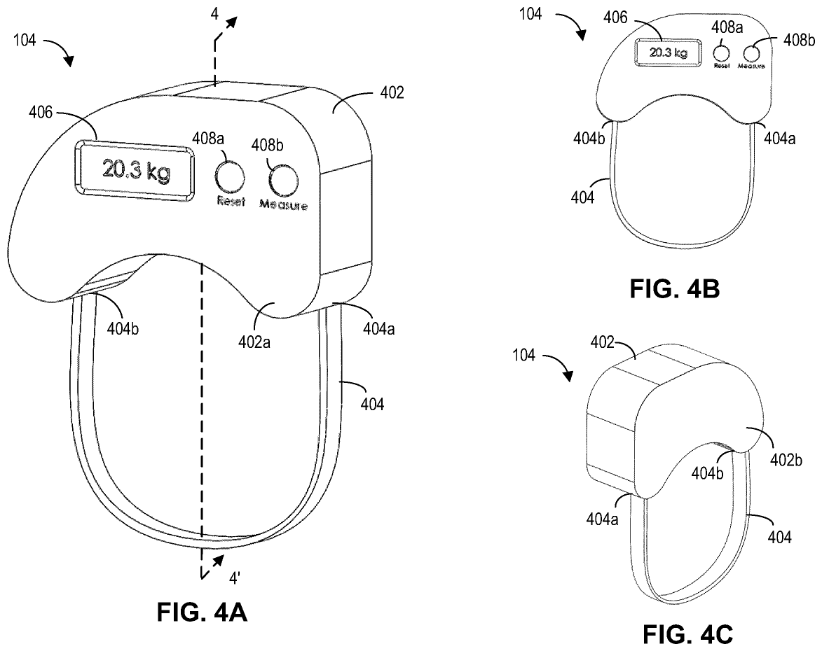

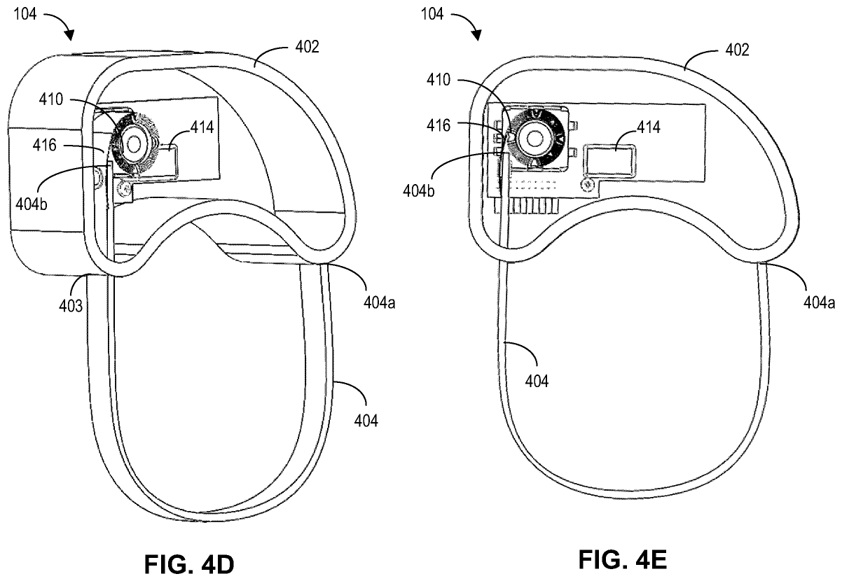

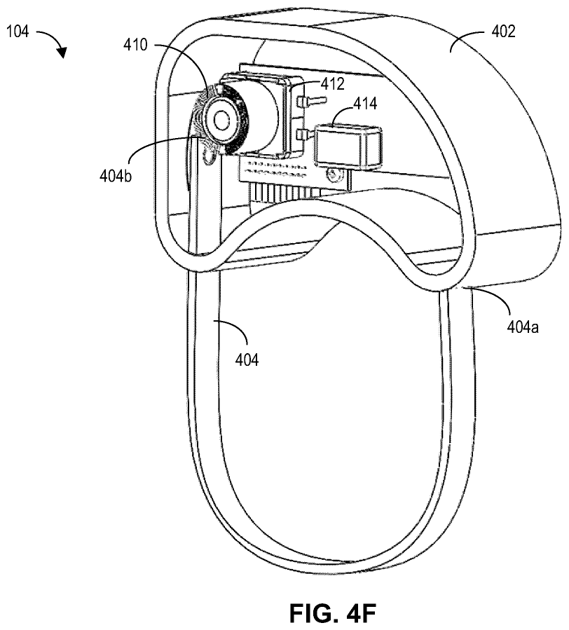

Reference is now made concurrently to FIGS. 4A-4F, which illustrate various schematic views of the weight estimation device 104, according to some embodiments.

As shown, in at least some example embodiments, the device 104 can include an outer casing 402, which can house various device electronics. In at least some embodiments, the outer casing 402 may be manufactured from a washable and/or durable material to enable re-use. In some cases, the material can be any one, or any combination, of plastic, metal, and synthetic materials.

Device 104 can also include a measuring strip 404. In a mounted or applied position, the strip 404 is wrapped around the patient's limb (i.e., 102a in FIG. 1), i.e., in a fitted engagement around the limb, to measure a limb circumference. In an unmounted position, the strip 404 may not otherwise be in fitted engagement around the patient's limb, i.e., the strip 404 may be loose around the limb. In the illustrated embodiment, the strip 404 may have a first end 404a, fixed to the outer casing 402, and a loose second end 404b receivable inside of the outer casing 402 via slit opening 403 (FIG. 4D).

In at least one embodiment, the strip 404 can be manufactured of a flexible material to facilitate circumference measurements. By way of non-limiting examples, the strip 404 can be manufactured of plastic, metal, paper, linen, natural or other manmade materials. In some cases, numbers and/or linear markings are printed on strip 404 to allow for rapid visual and sensor verification of digital measurements.

In the illustrated embodiment, the device casing may have one or more input and output interfaces, including a device display 406, a reset button 408a and a measure button 408b. In one example case, once the measuring tape 404 is securely wrapped around patient's limb, the measure button 408b can be selected. This, in turn, allows the weight measuring device 104 to automatically determine the patient's estimated weight. The estimated weight may then be displayed on the display 406. Alternatively, or in addition, the display 406 can display the measured circumference. If it is desired to acquire a new measurement, the user can simply select the reset button 408a. The input and output interfaces can be located on one or both of the front 402a and/or rear faces 402b of the device 104. Other additional buttons on the casing may also be provided to allow for automatic deployment of the measuring strip 404.

FIGS. 4D-4G illustrate various device electronics that may be housed inside the outer casing 402. As shown, the device electronics can include a rotatable spool 410 for receiving the loose second end 404b of the measuring strip 404. In some cases, a biased spring 416 (FIGS. 4E, 4F) may connect the spool 410 to the second tape end 404b to enable light application of circumferential pressure on the limb for more precise measurements. In some embodiments, the measuring strip is motor-operated, as for example by a motor operating the spool 410.

In the illustrated embodiment, spool 410 can connect to a rotary encoder 412. Rotary encoder 412 can measure or track the limb circumference based on the amount of measuring strip 404 spooled or unspooled from the rotating spool 410. The rotary encoder can be, by way of non-limiting examples, magnetic, optical, mechanical, or capacitive in nature.

In other cases, a linear encoder may be provided to measure limb circumference. The linear encoder can be, by way of non-limiting examples, optical, magnetic, inductive or capacitive in nature. In some cases, a combination of a linear and rotary encoder can be provided. For example, the linear encoder can provide absolute analog or digital data on large distance increments such as 10 cm. Conversely, finer, absolute or incremental length measurements can be assessed by a rotatory encoder. In an alternate embodiment, the encoding unit comprises a combination of several rotary encoders only. Similarly, another embodiment would comprise linear encoders and linear markings on the strip only. In various cases, multiple encoders can be used for redundancy and increasing measurement accuracy.

Additionally, a computing unit 414 may be provided which can include a processor, memory, and/or communication interface.

While not illustrated, the device electronics can include one or more sensors, such as pressure, force, or tactile sensors used to ensure constant pressure between measurements. In some cases, the sensor data (i.e., pressure data) may be displayed as well on the display interface 406.

In some embodiments, the digital weight estimation device provides additional patient data. Data such as blood pressure measurements, brachial pulse, bone density, body temperature, and oxygen saturation can also be provided. Similarly, additional features such as a cardiopulmonary resuscitation timer can be integrated in the weight-estimation device.

Reference is now made to FIG. 5, which shows a simplified block diagram of hardware for an example embodiment of a weight estimation device 104 and a computer terminal 106.

As shown, the weight estimation device 104 can include a processor 502a connected, via a data bus, to one or more of memory 504a, communication interface 506a, one or more encoder unit(s) 508a, one or more sensors 510a, a display interface 512a and one or more input interfaces 514a.

Processor 502a is a computer processor, such as a general-purpose microprocessor. In some other cases, processor 502a may be a field programmable gate array, application specific integrated circuit, microcontroller, or other suitable computer processor. In some cases, processor 502a may in fact comprise multiple processors.

Processor 502a is coupled, via a computer data bus, to memory 504a. Memory 504a may include both volatile and non-volatile memory. Non-volatile memory stores computer programs consisting of computer-executable instructions, which may be loaded into volatile memory for execution by processor 502a as needed. It will be understood by those of skill in the art that references herein to the weight estimation device 104 as carrying out a function or acting in a particular way imply that processor 502a is executing instructions (e.g., a software program) stored in memory 504a and possibly transmitting or receiving inputs and outputs via one or more interface. Memory 504a may also store data input to, or output from, processor 502a in the course of executing the computer-executable instructions. In at least some embodiments, memory 504a may store a weight estimation software 516a which can convert circumference data into estimated weight.

Communication interface 506a is one or more data network interface, such as an IEEE 802.3 or IEEE 802.11 interface, for communication over a network. In some cases, communication interface 506a may be configured for Bluetooth® communication.

Primary measurement sensor(s) 508a may be analogous to encoder units 412 (FIG. 4F). As discussed above, encoder unit (s) 508a may comprise one or more linear and/or rotary encoders for measuring limb circumference using measuring tape 404. In other cases, primary measurement sensor(s) 508a may comprise various types of motion sensors for tracking and/or recording movement of the measuring tape 404 into the mounted position, i.e., as it is being applied around the patient's limb.

Secondary sensor(s) 510a may include, as also stated previously, pressure, force, or tactile sensors for determining correct application of the weight measuring device 104 in the mounted position. Secondary sensors 510a can also include sensors for measuring blood pressures, brachial pulse, bone density, body temperature, and/or oxygen saturation, as well as broadly any other sensors for monitoring patient biometric data.

Display interface 512a may be analogous to display interface 406 (FIG. 4A), which is a suitable display for outputting information and data as needed (e.g., weight estimates). In some cases, display interface 512a comprises a liquid crystal display (LCD).

Input interface 514a may comprise any suitable interface for receiving user inputs, and may include buttons 408a, 408b in FIG. 4A.

Similar to weight estimation device 104, computer terminal 106 may also include processor 502b which is connected, via a data bus, to memory 504b, communication interface 506b, display interface 508b and input interface 510b. The components of the computer terminal 106 may be analogous in structure and function to the equivalent components as described in relation to the weight estimation device 104. As discussed, memory 504b may store the medication dose preparation software 512b.

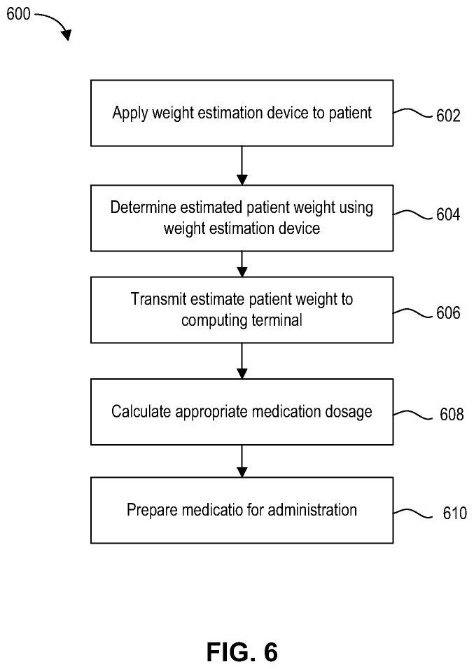

Reference is now made to FIG. 6, which shows a process flow for an example embodiment of a method 600 for medication dose preparation.

At 602, the weight estimation device 104 is applied to the patient's limb.

At 604, the weight estimation device 104 can estimate the patient's weight based on the limb circumference. For example, this may be performed using a circumference-based weight prediction algorithm. In various cases, the patient weight may be estimated and/or monitored in real-time or near real-time.

At 606, the patient's estimated weight may be transmitted to the external computing device 106, which host the medication dose software 512b. In other cases, the weight estimation device 104 can simply transmit the measured circumference data. In still other cases, weight estimation device 104 can transmit various sensor data associated with sensors located in the weight estimation device 104.

At 608, the medication dose software 512b—operating on the computing terminal 106—may calculate the appropriate medication dosage parameters. The dosage parameters may comprise various quantities associated with the medication dose to be administered to the patient based on the medication type, and one or more of the (i) the patient weight and (ii) other patient-specific data (i.e., patient age, gender, medical history, etc.). By way of non-limiting examples, the dosage parameters can include a suggest dosage of the medication, total dosage to administer of the medication, stock concentration of the medication, volume of medication to draw and volume of diluent. In various cases, the patient's estimated weight is determined based on data received from the weight estimation device 104. Further, the patient-specific data may be entered by a user into the medication dose software, or otherwise received externally, i.e., from server 210a.

At 610, the medication dose is prepared based on the determined medication dose parameters. In some cases, the medication dose preparation is performed manually, i.e., by a health care practitioner. In at least some embodiments, the medication dose software may provide guiding instructions to assist the practitioner in preparing the medication dose, as well as prioritizing between several types of medication to be administered. In other cases, act 610 may also be performed automatically. For example, the medication dose software 512b may be operable to control an automated bolus dose preparation system 110 (FIG. 1B).

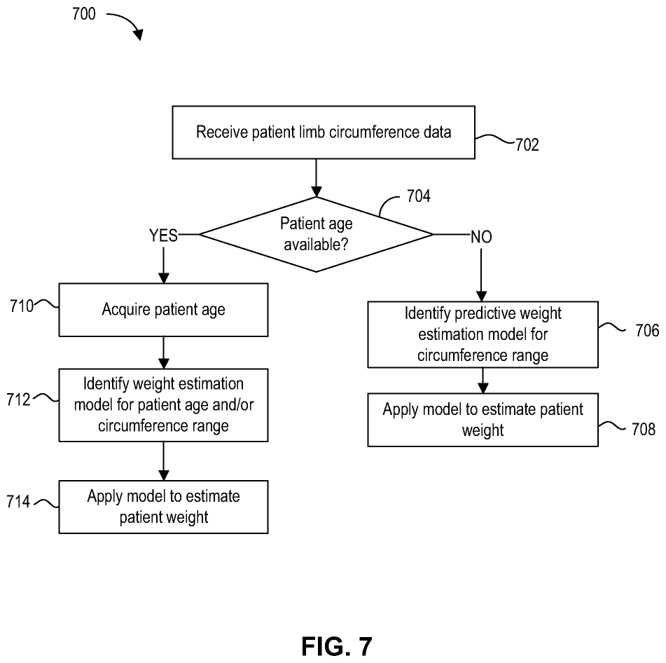

Reference is now made to FIG. 7, which shows a process flow for an example embodiment of a method 700 for automated weight estimation (i.e., act 604 in method 600). Method 700 can be performed using one or more of the processor 502a of the weight estimation device 104 and/or the processor 502b of the computer terminal 106.

At 702, the weight estimation software 516a—operating on either the weight estimation device 104 or computer terminal 106—may receive patient limb circumference data. For example, the limb circumference data may be generated by the weight estimation device 104 applied around the patient's limb, as previously described.

At 704, a determination is made as to whether the patient's age is available. As provided herein, the patient's age may be used to enhance the predictive accuracy of the weight estimation model.

If the patient's age is not available, then method 700 may proceed to acts 706 and 708. At 706, the weight estimation software 516a can identify a suitable predictive weight estimation model based on the measured limb circumference. In at least one embodiment, different predictive models may be selected for different limb circumference ranges. For example, a first predictive model can be used for limb circumferences in a first range (i.e., below 15 centimeters (cm)), a second predictive model can be used for limb circumferences in a second range (i.e., between 15 cm and 30 cm), and a third predictive model can be used for limb circumferences in a third range (i.e., above 30 cm). To this end, it has been appreciated that, to increase weight prediction accuracy—rather than relying on a single predictive model, a combination of predictive models can be used to accommodate for variations in limb circumferences. In particular, each predictive model may comprise a combination of linear and non-linear functions that relate limb circumference to patient weight.

At 708, the limb circumference data may be applied to the selected weight estimation model to generate an estimate of the patient's weight.

Returning to act 704, if the patient's age is available—at 710, the weight estimation software 516a can acquire the patient's age. In some cases, act 706 may be performed prior to act 704 such that if the software is unable to acquire the patient's age, the method may automatically revert to acts 706 and 708.

In at least one embodiment, at 710, the patient's age is manually acquired. For instance, the input interface 514a to the weight estimation device 104—or the input interface 510 to the computer terminal 104—may allow a user (i.e., medical practitioner) to manually input the patient's age.

In other embodiments, the patient's age may be acquired through more automated methods. For example, a scanning system may couple to either the weight estimation device 104 and/or computer terminal 106. The scanning system can comprise, for example, a camera or a barcode reader. The scanning system may be employed to scan (or read) a scannable indicium associated with the patient. For example, this may be a scannable indicium, i.e., a barcode or QR code, located on a patient identification wristband.

In at least one case, the scanning system may scan the indicia to automatically acquire the patient's age. In other cases, the scanning system may scan the indicia to retrieve secondary information, such as a patient ID number. The weight estimation software 516a can then cross-reference the acquired patient ID to a database storing patient-specific data, i.e. including the patient's age. The cross-referenced database may be a local database stored on the weight estimation device memory 504a and/or computing terminal memory 512b. In other cases, the database may be externally hosted on one or more of servers 210a, 210b. In still other cases, the patient ID—if known—may also be manually input by a user into the input interfaces 514a, 514b.

At 712, the weight estimation software 516a may identify a suitable weight estimation model based on both the measured limb circumference and the patient's age.

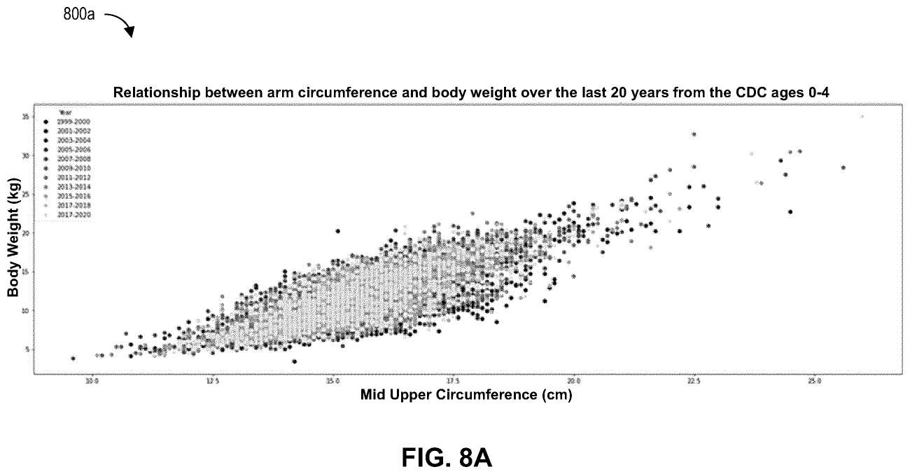

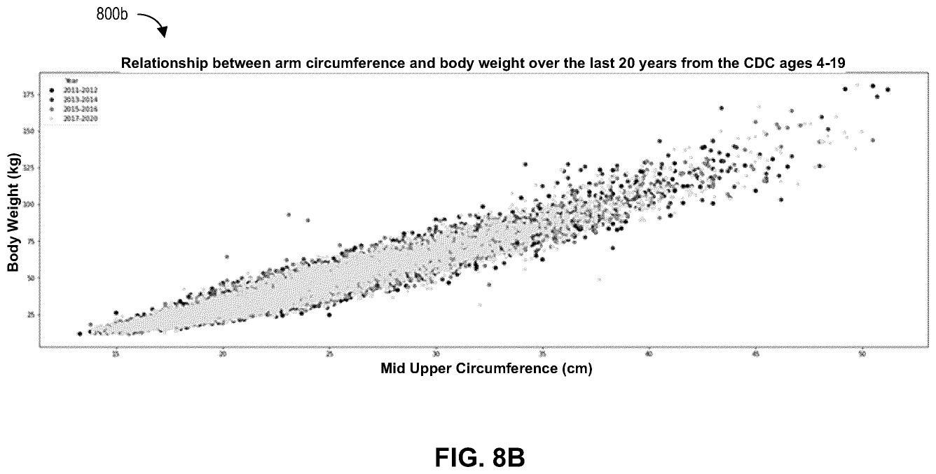

In at least one embodiment, different estimation models may be used based on the patient's age range. For example, FIGS. 8A, 8B and 8C show plots 800a, 800b, and 800c respectively, illustrating the correlation between arm circumference (centimeters) and body weight (kilograms) for subjects within the age ranges of 0 to 4 years old (FIG. 8A), 5 to 7 years old (FIG. 8B) and 8 to 19 years old (FIG. 8C). Equation (1) shows a linear regression model generated based on plot 800a, while Equations (2) and (3) show linear regression models generated based on plot 800b and 800c, respectively.

$$Weight = {1.25991537}^{*} ( Arm Circumference ) + {1.66472123}^{*} ( Age ) - \text{.10} \text{.054796602458609} \qquad ( 1 )$$

$$Weight = {2.0528604}^{*} ( Arm Circumference ) + {1.39655423}^{*} ( Age ) - 23.24261052651997 \qquad ( 2 )$$

$$Weight = {3.19321569}^{*} ( Arm Circumference ) + {1.01703969}^{*} ( Age ) - 42.74004008992316 \qquad ( 3 )$$

Equation (1) yields an average predictive error of approximately 8.04%, and an R² value of approximately 0.90. Equation (2) yields an average predictive error of approximately 6.73%, and an R² value of approximately 0.89. Equation (3) yields an average predictive error of approximately 7.84%, and an R² value of approximately 0.92. Tables 1, 2 and 3, below, illustrate the percentage agreement between the expected and predicted data using Equation (1) (Table 1), Equation (2) (Table 2), and Equation (3) (Table 3).

TABLE 1
Percentage in agreement within

| 10% | 20% | 30% | 40% | 50% | 60% | 70% | 80% | 90% | 100% | >100% |
|---|---|---|---|---|---|---|---|---|---|---|
| 70.4% | 93.8% | 98.8% | 99.7% | 100% | 100% | 100% | 100% | 100% | 100% | 100% |

TABLE 2
Percentage in agreement within

| 10% | 20% | 30% | 40% | 50% | 60% | 70% | 80% | 90% | 100% | >100% |
|---|---|---|---|---|---|---|---|---|---|---|
| 77.1% | 97.9% | 99.8% | 99.9% | 99.9% | 100% | 100% | 100% | 100% | 100% | 100% |

TABLE 3
Percentage in agreement within

| 10% | 20% | 30% | 40% | 50% | 60% | 70% | 80% | 90% | 100% | >100% |
|---|---|---|---|---|---|---|---|---|---|---|
| 69.4% | 95.9% | 99.6% | 99.9% | 99.9% | 99.9% | 100% | 100% | 100% | 100% | 100% |

Accordingly, a two-feature predictive model (i.e., based on age and weight) is expected to generate higher accuracy weight predictions.

In at least one embodiment, within each age group, separate predictive models are provided for different circumference ranges, as explained with reference to act 706. For example, for a given age group: a first age-specific predictive model can be used for limb circumferences in a first range (i.e., below 15 centimeters (cm)), a second age-specific predictive model can be used for limb circumference in a second range (i.e., between 15 cm and 30 cm), and a third age-specific predictive model can be used for limb circumferences in a third range (i.e., above 30 cm). Accordingly, at 712, identifying the correct weight estimation model may involve: (a) initially, identifying the appropriate age range; and (b) subsequently identifying the appropriate circumference range.

At 714, the selected predictive model is applied to estimate patient weight.

In some cases, the weight estimation device 104 may be used to measure circumferences for different types of limbs (i.e., mid upper arm, thigh, calf etc.). In these cases, the weight estimation device 104 may enable users to select the particular limb type to measure (i.e., via the input interface 514a). At 702, the weight estimation software 516a may receive the limb circumference data, as well as an indicator of the limb type associated with the circumference data. At 706 or 712, the weight estimation software 516a may identify the set of predictive models that are applicable to that limb type (i.e., based on limb circumference range and/or patient age). Accordingly, the predicted weight may be tailored for the measured limb.

The present invention has been described here by way of example only, while numerous specific details are set forth herein in order to provide a thorough understanding of the exemplary embodiments described herein. However, it will be understood by those of ordinary skill in the art that these embodiments may, in some cases, be practiced without these specific details. In other instances, well-known methods, procedures and components have not been described in detail so as not to obscure the description of the embodiments. Various modification and variations may be made to these exemplary embodiments without departing from the spirit and scope of the invention, which is limited only by the appended claims.

1.-25. (canceled)

26. A system for weight-based medical dose preparation, the system comprising:
a weight measuring device comprising:
a measuring strip configured to be applied around a patient's limb, the measuring strip movable between an unmounted position and a mounted position, in which in the mounted position, the strip engages around the patient's limb;
a primary measurement sensor for measuring limb circumference using the measuring strip; and
a device processor coupled to the primary measurement sensor, the device processor operable to:
determine, based on data received from the primary measurement sensor, a limb circumference;
estimate a patient weight based on the limb circumference; and
transmit the patient weight to a computing terminal,
the computing terminal comprising a computing processor operable to:
receive the patient weight from the weight measuring device;
determine, based on the given medication type and one or more of the patient weight and patient-specific data, one or more dosage parameters; and
output the one or more dosage parameters.

27. The system of claim 26, wherein the weight measuring device is located remotely from the computing terminal, and the system further comprises a communication network, and wherein,
the weight measuring device further comprises a device communication interface coupled to the device processor, and the device processor being further configured to transmit the patient weight over the communication network, via the device communication interface; and
the computing terminal further comprises a computing communication interface coupled to the computing processor, and the computing processor is operable to receive the patient weight via the computing communication interface.

28. The system of claim 27, wherein the communication network comprises a personal area network (PAN).

29. The system of claim 26, wherein the weight measuring device and the computing terminal are directly coupled to form a single integrated device.

30. The system of claim 26, wherein the primary measurement sensor comprises an encoder unit.

31. The system of claim 26, wherein the measuring strip is applied around one of the patient's middle-upper arm and the patient's thigh.

32. The system of claim 26, wherein the dosage parameters comprise one or more of a suggested dose, a total dose, and a drug volume.

33. The system of claim 26, wherein the patient-specific data comprises one or more patient age and patient gender.

34. The system of claim 26, wherein estimating the patient weight based on the limb circumference comprises:
identifying a circumference range for the limb circumference; and
applying a predictive model associated with the identified circumference range.

35. The system of claim 34, wherein estimating the patient weight based on the limb circumference comprises:
determining a patient age range; and
applying the predictive model associated with the identified circumference range and the patient age range.

36. A method for weight-based medical dose preparation for a given medication type, comprising:
determining, based on data received from a primary measurement sensor of the weight measuring device, a limb circumference, wherein the primary measurement sensor measures limb circumference using the measuring strip;
estimating a patient weight based on the limb circumference;
determining, based on the given medication type and one or more of the patient weight and patient-specific data, one or more dosage parameters; and
outputting the one or more dosage parameters.

37. The method of claim 36, wherein the method further comprises:
transmitting the patient weight over a communication network from the weight measuring device to a remote computer terminal; and
receiving the patient weight at the computing terminal.

38. The method of claim 37, wherein the communication network comprises a personal area network (PAN).

39. The method of claim 37, wherein the weight measuring device and the computing terminal are directly coupled to form a single integrated device.

40. The method of claim 36, wherein the primary measurement sensor comprises an encoder.

41. The method of claim 36, wherein the measuring strip is applied around one of the patient's middle-upper arm and the patient's thigh.

42. The method of claim 36, wherein the dosage parameters comprise one or more of a suggested dose, a total dose, and a drug volume.

43. The method of claim 36, wherein the patient-specific data comprises one or more of patient age and patient gender.

44. The method of claim 36, wherein estimating the patient weight based on the limb circumference comprises:
identifying a circumference range for the limb circumference; and
applying a predictive model associated with the identified circumference range.

45. The method of claim 44, wherein estimating the patient weight based on the limb circumference comprises:
determining a patient age range; and
applying the predictive model associated with the identified circumference range and the patient age range.

46.-50. (canceled)
